(19) United States
(12) Patent Application Publication
(10) Pub. No.: US 2012/0024743 A1
(43) Pub. Date: Feb. 2, 2012

(54) TRANSDERMAL PHARMACEUTICAL PREPARATIONS

(75) Inventors: Endre Mikulasik, Alsonemesapati (HU); Patrik Fazekas, Kormend (HU)

(73) Assignee: EGIS GYOGYSZERGYAR NYILVANOSAN MUKODO RESZVENYTARSASAG, BUDAPEST (HU)

(21) Appl. No.: 13/148,219

(22) PCT Filed: Feb. 5, 2010
(86) PCT No.: PCT/HU2010/000015

§ 371 Date: Oct. 21, 2011

(30) Foreign Application Priority Data
Feb. 6, 2009 (HU) P0900072

(51) Int. Cl. B65D 85/00; A61K 31/522; A61K 31/5415; A61K 31/192; A61K 31/196; A61K 31/4164; A61K 31/4418; A61K 31/21; A61K 31/495; A61K 31/167; A61K 31/515; A61K 31/505; A61K 38/38; A61K 33/04; A61K 31/472; A61K 31/4184; A61K 31/46; A61K 31/702; A61K 31/573; A61K 31/42; A61K 31/4152; A61P 25/00; A61P 9/00; A61P 11/00; A61P 1/16; A61P 3/00; A61P 15/00; A61P 17/00; A61P 37/02; A61P 19/00; A61P 25/16; A61P 25/18; A61P 29/00; A61P 25/26; A61P 31/04; A61P 31/10; A61P 31/12; B05D 7/00; B05D 5/00; A61K 9/14

(52) U.S. Cl. 206/524.1; 424/490; 514/263.38; 514/226.5; 514/570; 514/567; 514/396; 514/398; 514/356; 514/509; 514/255.04; 514/626; 514/270; 514/275; 514/15.2; 424/705; 514/307; 514/395; 514/304; 514/61; 514/179; 514/380; 514/404; 514/629; 514/263.34; 427/2.1

(57) ABSTRACT

The present invention relates to semisolid transdermal pharmaceutical preparation having enhanced stability and bioavailability, wherein the particles are coated by a volatile silicon oil component and the thus obtained suspension is dispersed in a gel or cream base.

### TECHNICAL FIELD OF THE INVENTION

The present invention relates to semisolid transdermal pharmaceutical preparations, which comprise coated particles of the active ingredient dispersed in a gel- or cream base and method for preparation thereof. More particularly, the invention is related to formulations intended for transdermal use, wherein the active ingredient is coated by volatile silicones (siloxanes) and the thus obtained suspension is dispersed in gel or cream vehicle base. Physical, chemical and microbiological stability of the transdermal formulations according to the present invention are excellent, manufacture thereof can be carried out by simple operations and by selecting appropriate volatile silicone constituents for coating the active ingredient, it has been possible to produce transdermal preparations for topical, local or systemic use.

### TECHNICAL BACKGROUND OF THE INVENTION

Due to excellent chemical inertness, resistance against heating and cooling, compatibility with biological systems as well as excellent mechanical properties depending on the chemical structure, the application area of the silicones (also referred to as siloxanes, silanes or polysiloxanes) is especially wide. Silicones can contain a linear polysiloxane chain (e.g. silicone oils, caoutchoucs), cyclic or branched chain (e.g. silicone resins) or of reticular structure having a molecular weight up to 700.000 daltons. Siloxanes are usually applied in the pharmaceutical industry as silicone oils of different viscosity.

The boiling point and viscosity of the silicone oils are principally determined by their degree of polymerization. Silicone derivatives having lower degree of polymerization are free flowing, volatile liquids. The boiling point and viscosity are increasing with increasing degree of polymerization. Above a critical degree of polymerization or by the formation of reticular structure due to cross-binding, the silicones are presented as semisolid or solid elastic materials, e.g. silicone caoutchoucs and silicone gums.

Polysiloxanes are principally produced by hydrolysis of partially alkyl-substituted halogen-silanes or mixtures thereof. For example, according to European Patent No. 980885, mixture of trimethylchlorosilane and dimethyl-dichlorosilane are hydrolized in the presence of aqueous hydrochloric acid solution, thereby obtaining mixtures of silicone polymers, which are refined by distillation and fractionation.

The introduction of silicones in the medicine was delayed by the fact that the production of these compounds was especially costly and complicated in the quality necessary for the purposes of the medicine. For example, silicone oils intended for the use in the ophtalmology were often found to contain monomers or oligomers, which degraded the suitability of the oil for the intended purpose and were found to be potentially harmful to the health. Silicone polymers are used in the medicine for the purpose of medicated and surgical implants, prostheses and in medical devices.

High-volatility silicones belong to the group of silicone oils. Under the expression of “volatile silicone” are meant those silicone oils used as pharmaceutical auxiliary agents, which are avaporated from the human skin within less than six hours and do not leave any residue thereafter. Such volatile silicones can be produced in the quality suitable for the manufacture of medicaments.

The use of silicones of various degree of polymerization for the formulation of cosmetic and pharmaceutical preparations as well as in nutrient formulations is known from the state of the art. Silicone oils and caoutchoucs of higher molecular weight are usually applied as vehicle, film forming agent, while silicone oils have been used as dispergants or stabilizers in the state of the art.

Volatile silicones are used according to the state of the art for dispersing partially miscible liquids or solids in a continous liquid phase in cosmetic or pharmaceutical emulsions or suspensions. The formulations of European Patent No. 639372 are cosmetic or pharmaceutical aerosols, wherein hexamethyldisiloxane is used as dispersing agent for the homogenization of the active ingredient, tixotropic auxiliary agent and solid vehicle, e.g. talc.

The use of some volatile silicones as vehicle in cosmetic preparations has been disclosed in European Patent Application No. 1472263.

British Patent No. 2064363 discloses a liquid vehicle system which is suitable for enhancing the penetration into the upper epidermis layer of the skin comprising water, a volatile silicone and an emulsifying agent selected from an ethoxylated fatty acid or an ethoxylated sorbitane ester. A similar preparation containing vitamine D as pharmaceutically active ingredient has been disclosed in International Patent Application WO2005053666 wherein an additional non-volatile hydrocarbon or ester is used as component of the vehicle.

Published International Patent Application WO2006100489 discloses a liquid formulation presented as an emulsion, which comprises an active ingredient, a penetration enhancing agent, a penetration modulating agent, and volatile vehicle. Among penetration enhancing agents, benzylalcohol, among penetration modulating agents volatile silicones are mentioned. The vehicle is a mixture of short-chain alcohols. The preparation is suitable for administering pharmaceutically active ingredients intended for systemic effect.

The drawback of liquid pharmaceutical preparations resides in the fact that due to the liquid state, the period of application and the applied dose is poorly repeatable and reproducible. Thus, such preparations, even in cases when the period of application is short, can be recommended for topical or local applications (e.g. skin, mucosa, muscular system below the skin and in the vicinity of the application area) only.

Volatile silicones are rarely used is semisolid pharmaceutical preparations. European Patent No. 410099 discloses water-free antibacterial gels for topical use, wherein the active ingredient is a tetracycline antibiotic and the vehicle consists a silicone component or mixture selected from octamethylcyclotetrasiloxane, decamethylcyclo-pentasiloxane or hexamethyl-disiloxane or mixtures thereof, a polymer selected from acrylates, vinylacetate or polyethylene homopolimers as gelling and film-forming agent and an ester-type softener.

European Patent No. 980 885 discloses cosmetic preparations containing the cosmetic ingredients dispersed in a gel comprising a volatile silicone dispersing agent, a non-volatile paraffin, water and hydroxypropymethylcellulose.

European Patent No. 998 943 discloses an essentially water-free gel formulation consisting of octamethylcyclotetrasiloxane, decamethyl-cyclopentasiloxane, hexamethyldisiloxane or mixtures thereof, vitamine E and hydrogenated castor oil.

U.S. Pat. No. 4,355,046 and U.S. Pat. No. 5,336,692 disclose the use of hexamethyldisiloxane, octamethyltrisiloxane and decamethylpentasiloxane for the homogeneous distribution of the cosmetic preparation on the skin surface.

Published International Patent Application No. WO2009007764 disclose a transdermal formulation with improved absorption and bioavailability containing acyclovir, piroxicam, meloxicam, ibuprofen, diclofenac sodium or potassium, clotrimazole, bifonazole, metronidazole, nifedipine, nitroglycerol or cetirizine as an active ingredient, containing suspension of particles of the active ingredient in volatile silicones, said suspension being dispersed in a gel or cream base.

There is a need for methods of administration for pharmaceutically active ingredients which is non-invasive and can be used in those cases where ingestion of a tablet is difficult, e.g. in the case of elderly people or infants. Methods of administration bypassing the enteric route are also desirable for those pharmaceutically active ingredients which are prone to metabolism at the place of absorption in the enteric system or undergo extensive first-pass metabolism.

According to the state of the art, there are no known pharmaceutical formulations in the form of transdermal creams or gels containing a volatile silicone or mixture of such compounds which provides for the systemic effect of the active ingredient.

The advantage of the transdermal application route for achieving systemic effect resides in the fact that the concentration profile of the active ingredient in the blood plasma is steady. Furthermore, the transdermal method of application method is suitable for the introduction of active ingredients into the body which are absorbing poorly from the enteric system, irritating, eliminated rapidly or inactivated instantly during their metabolism. The main drawback of the transdermal application method resides in the fact that patches or creams may cause irritation, alterations of the skin and in some cases, their removal may be difficult or may not be removed from the application area in full.

The disadvantage of lipophilic creams known from the prior art resides in the fact that the absorption of the active ingredient is poor and slow, because due to the distribution of the lipophilic vehicle and the outer layer of the skin, the greatest part of the active ingredient remains in the constant-volume vehicle.

Hydrophilic gel formulations containing the active ingredient in suspended state are known from the state of the art. Although the absorption from such formulations is in most cases sufficient, these preparations are prone to physical-chemical alterations during storage including decomposition of the active ingredient, degradation of the colloidal structure of the formulation and often microbiological contamination occurs. Such processes diminish the stability and shelf life of the preparation.

The principal requirement for transdermal pharmaceutical formulations including semisolid gel and cream preparations is stability, sufficiently long shelf life, sufficient absorption of the active ingredient for the therapeutical application and appropriate physical state under the circumstances of the application.

### SUMMARY OF THE INVENTION

The present invention provides semisolid transdermal pharmaceutical preparations in the form of gels or creams, wherein the gel or cream base serving as vehicle contains dispersed particles of the active ingredient coated by high-volatility silicone oil or by a mixture thereof. In the preparations according to the present invention, the most preferably, hexamethyldisiloxane, octamethyltrisiloxane or decamethyl-pentasiloxane can be used. The transdermal semisolid preparations according to the present invention are suitable for application to the skin or a mucous membrane optionally in form of dosage units and it is possible to produce the transdermal composition according to the present invention in a form which allows the development of topical, local or systemic effect, depending on the composition. Compositions according to the present invention possess excellent physical-chemical and microbiological stability.

### DETAILED DESCRIPTION OF THE INVENTION

The objective of our research was to develop a transdermal semisolid pharmaceutical dosage form, which is suitable for the formulation of pharmaceutically active, cosmetic or nutritional ingredients with good absorption, penetration and bioavailability, while at the same time, shows appropriate physical chemical stability, devoid of microbiological contamination or decomposition and has appropriately long shelf life. Furthermore, we intended to develop a vehicle system, which can be formulated to achieve reproducible targeted delivery of the desired component of the formulation to the place where the therapeutic effect is desired, including the possibility of obtaining topical, local or systemic effect.

The above objective is achieved according to the present invention.

Surprisingly, we have found that by using volatile silicones as auxiliary agent, a semisolid transdermal preparation can be produced which satisfy the above-mentioned requirements. The stability, absorption and penetration properties of creams and gel is governed by the quality and proportion of the volatile silicone or the mixture thereof.

The expressions “silicone”, “silane” and “siloxane” are used interchangeably throughout the present specification and represent compounds of the element silicone wherein the silicone atoms in the polysiloxane O—[SiR¹R²—O]ₙ—Si chain are substituted by R¹, R² alkyl groups.

In the present specification, the expression “transdermal formulation” represents any pharmaceutical preparation, which is applied to the skin, independently from that the pharmacological effect is manifested at the application area of the preparation, in the tissues located in the vicinity thereof or throughout the whole body including organs and tissues located far from the place of the application.

Accordingly, the expression “topical effect” means that the pharmacological effect occurs exclusively at the area whereto the transdermal formulation according to the present invention is applied.

The meaning of the expression “local effect” is that the pharmacological effect occurs in tissues located in the close vicinity of the area where the transdermal formulation according to the present invention is applied to. For example, topical preparation applied to the skin may exert its effect in the muscular system under the skin but the active ingredient is either undetectable in the blood plasma or the concentration thereof is far less than that necessary for therapeutical action.

The expression “systemic effect” represents that the pharmacological effect occurs throughout the whole body or organism, even in tissues or organs located distantly from the area of the application where the transdermal formulation according to the present invention is located. The active ingredient from such preparations usually is absorbed from the area of application into the bloodstream.

According to the first aspect of the present invention, there are provided transdermal pharmaceutical preparations, which comprise particles of the active ingredient admixed or coated with one or more volatile siloxane dispersed in a cream or gel base.

It has been recognized unexpectedly that the transdermal semisolid preparations according to the present invention are suitable for application to the skin or a mucous membrane even in form of dosage units and it is possible to produce the transdermal compositions according to the present invention in a form which allows the development of topical, local or systemic effect, depending on the composition. This effect is very surprising, since it has not been possible so far according to the state of the art to achieve systemic effect by a semisolid transdermal formulation.

According to the second aspect of the present invention, there are provided transdermal pharmaceutical preparations suitable for topical use, which comprise particles of the active ingredient admixed or coated with one or more volatile siloxane dispersed in a cream or gel base. In the context of the present application, the expression “topical effect” means that the pharmacological effect occurs exclusively at the skin area where the transdermal formulation according to the invention is applied to.

According to the third aspect of the present invention, there are provided transdermal pharmaceutical preparations suitable for achieving local effect, which comprise particles of the active ingredient admixed or coated with one or more volatile siloxane dispersed in a cream, ointment or gel base. Under the expression of “local effect” is meant that the pharmacological effect occurs in tissues located in the close vicinity of the area where the transdermal formulation according to the present invention is applied to.

According to the fourth aspect of the present invention, there are provided transdermal pharmaceutical preparations suitable for obtaining systemic effect, which comprise particles of the active ingredient admixed or coated with one or more volatile siloxane dispersed in a cream, ointment or gel base. Under the expression “systemic effect” is meant that the pharmacological effect occurs throughout the whole body or organism, even in those tissues or organs which are located distantly from the area of the application where the transdermal formulation according to the present invention is located. The active ingredient of the preparation according to this aspect of the invention is usually detectable in blood plasma.

The person skilled in the art will, however, appreciate that it is not possible to distinctly separate the three class according to the primary site of therapeutical effect. It is known that slight absorption of the pharmaceutically active ingredient occurs even in the case of topical formulations, although usually this is not desired or intended. Furthermore, it may occur that an active ingredient of a formulation intended for local effect enters into the blood circulation and to some less degree, systemic effect occurs although this is not intended. It is therefore possible to devise formulations according to the present invention, which are intermediary according to their site of action, i.e. they act topically and locally or, locally and systemically. This multiple action is, however, sometimes advantageous since it may enhance the therapeutical effect. For example, in case of antifungals, it is advantageous to treat the fungal infection at the skin surface and to some extent, in deeper layers of the skin and skin appendices (which amounts to local effect). Therefore, a targeted drug delivery can be achieved.

A particularly advantageous and surprising effect of the present invention that transdermal preparations suitable for administration through the skin can be prepared which allow the active ingredient to be absorbed from the skin in such a high degree that penetration into the circulation becomes possible, thereby providing for systemic effect. The rate of absorption of such preparations may be comparable to that achieved by oral administration without the possible difficulties of ingesting a tablet. It is possible to deliver dosage units of the transdermal formulation corresponding to the usual oral dose (or a blood plasma level achieved by the administration of the usual oral dose) to the skin.

In the formulation according to the present invention, the volatile silane component is preferably selected from hexamethyldisiloxane, octamethyltrisiloxane, decamethylpentacyclosiloxane or mixtures thereof. However, other volatile silicones can also be used. As a base vehicle, preferably a gel-forming polymer, such as a carboxyvinyl polymer, hydroxypropylmethylcellulose, methylcellulose or like or a mixture of such can be used.

The composition according to the present invention can contain one or more active ingredients. The scope of the active ingredients is not limited particularly to pharmaceutically active ingredients and cosmetic ingredients, but may include other chemicals applied to the skin of humans or animals (e.g. insecticides). The active ingredient can exert its effect topically, locally or systemically. It is understood that some active ingredients may find only external use and these are usually formulated as a preparation for topical administration. Those active ingredient which can be used externally or internally, can be formulated either for topical, local or systemic therapeutical effect depending on the therapeutical aim.

However, physical-chemical properties of the active ingredients also influence their applicability in the formulations according to the present invention. It has been found that those active ingredients which are present in aqeuous solution in mostly dissociated form, which are swelling significantly or which are strong bases or acids could not be easily formulated according to the present invention.

There is no explicite limitation regarding the pharmaceutically active ingredients which can be used in the transdermal formulations according to the present invention. For example, the active ingredient can be useful for the treatment or prevention of an infenctious disease, a cancerous or hematological disease, a disease belonging to the group of endocrinological, nutritional or metabolic disorders, a disease of the central nervous system, a disease due to malnutrition, a psychiatric disease, a behaviourial disorder, a compulsive disorder, a sexual or sexually transmitted disease, diseases and conditions of the mental and cognitive function, neurological diseases, stroke, ophtalmological diseass, an otolaryngological disease, a cardiovascular or cerebrovascular disease, a disease of the respiratory organs, a pulmonological disease, a dental disease, a disease or disorder belonging to the field of gastroenterology or hepatology. Active ingredients usually applied in dermatology, immunology, andrology, gynaecology and obstetrics, for the treatment of the diseases of the bone-arthritic and muscular system can be formulated according to the present invention. The formulation according to the present invention can be very advantageously used for the preparation of medicines against external physical effects or biological agents including but not limited to burns, frostbites, microbiological, against animal or herbal poisons and toxins, internal or external parasites or microorganism-caused infections or for the acceleration of wound healing and to relieve allergic reactions. It is also possible to formulate diagnostics or disinfectants according to the present invention.

The pharmaceutically active ingredient of the present invention can be selected from those suitable for the treatment of the nervous system including analgesics, anaestetics, antipyretics, anti-migraine, hypnotic, sedating, antidepressant, anxiolytic, antipsychotic, antiparkinson, antiepileptic, tranquillant or anticonvulsive ingredients, e.g. lidocaine, tetracaine, procaine, benzocaine, phenobarbital, thiopental, hexobarbital, a compound belonging to natural or synthetic opioid derivatives, amidazophen, novamidazophen, paracetamol, aspirine, theophilline, caffeine, alprazolam, an oxazepine tiazepine or diazepine derivative, a benzodiazepine, a phentiazine or indole derivative, an oxypropaneamine derivative, a diphenylamine derivative, zolpidem, risperidone, aripiprazole, olanzapine, ondansetron; donepezil, granisetron, metamizole, aminophenazon, phenacetin, ergotamine, naratriptane or another selective serotonine agonist, a monoamine or serotonine reuptake inhibitor, a cholinesterase inhibitor or a stimulant.

The active ingredient formulated according to the present invention can also be selected to be effective against the diseases of the cardiovascular or haematological system. For example, the formulation can contain an anticoagulant, antihypertensive, antilipemic, alpha or beta adrenoreceptor antagonist, platelet aggregation inhibitor, antisclerotic, ion channel blocking, antiarrhytmic, vascular dilating or thrombolytic agent, e.g. a cardiac glycoside, troxerutine, nitroglycerol, pentaerithritol-tetranitrate, tetranitrate, isosorbid-nitrate, nifedipine, amlodipine, felodipine, verapamil, diltiazem, an ACE-inhibitor, including captopril, perindopril, enalapril, ramipril or lisinopril, an angiotensin II-inhibitor, including to valsartan, losartan, irbesartan, olmesartan or telmisartan, a coumarine derivative, a heparin derivative, a trombocite aggregation inhibitor including clopidogrel, ticlopidine, prasugrel and acetylsalicylic acid or ibuprofen, a thrombin inhibitor, a stypic-adstringent agent, methyldopa, prazosin, doxazosin, terazosin, hydralazine, alprenolol, propranolol, metoprolol, bisoprolol, atenolol, nebivolol, carvedilol, nicotinic acid, pentoxyphilline, ergot alcaloids or bencyclane.

As an active ingredient effective against inflammation and suitable for acting at the immune system, an antiinflammatory, antihistaminic, immunesupressant, immune stimulating, antiallergic, antirheumatic, immune modulating, antiarthritic, leucotriene antagonist compound or a antigen suitable for inducing immune response can be used. Such compounds are e.g. benzydamine, salicylic acid derivatives, heparine derivatives, bioflavonoids, non-steroidal antiinflammatory drugs including diclofenac and its salts, ibuprofen, ketoprofen, flurbiprofen; and prostaglandin-derivatives.

Among the pharmaceutically active ingredients suitable against infections, a general disinfectant, an antibiotic, a chemotherapeutical agent, an antimicrobial, antibacterial, antifungal or antiviral compound or an antigen suitable for inducing immune response against an infenctious agent can be used. Examples for active ingredients suitable against infections are trimethoprim, sulfadimidine, sulfamethoxazole, econazole, miconazole, clotrimazole, ketoconazole, terbinafine, tolnaphtate, acyclovir, ribavirine, gancyclovir, valacyclovir, lamivudine, epervudine, neomycine and other aminoglycoside antibiotics; macrocyclic antibiotics, chlarithromycin, erythromycin, tylosine; tetracycline or fluoroquinolone type antibiotics. Examples of a general disinfectant are hydrogen peroxide or a complex thereof, benzoyl peroxide, cetylpyridinium, cetrimonium or tetraalkylammonium derivatives, triclosan, benzotrimethylammonium derivatives, lactic acid derivatives and chlorhexidine. The composition according to the present invention can contain an active ingredient effective against external or internal parasites as well as an insecticide.

In the formulation according to the present invention, non-steroid or steroid antiinflammatory compounds can also be advantageously used, e.g. hydrocortisone, prednisolone, methylprednisolone, triamcinolone, betamethasone, budenoside, dexamethasone, fluocinolone, diclofenac, ibuprofen, flurbiprofen and ketoprofen.

Examples of active ingredients useful for the treatment of the digestive and secretory system are diuretics, choleretics, antiulcer, antacid, antiemetic, appetite reducing, adstringent or laxative compounds, e.g. cimetidine, ranitidine, famitidine, cisapride, omeprazole, pantoprazole, lansoprazole, rabeprazole, esomeprazole, albumin tannate, pancreatine, trypsine, bromelaine, papaverine, drotaverine, atropine, hyoscyamine, belladonna alkaloids and derivatives thereof, deoxycholic acid derivatives, sylimarine derivatives, phenolphtalein, sibutramine, rimonabant, hydrochlorothiazide, chlorothiazide, teobromine, furosemide, spironolactone, amiloride and triamterene.

The transdermal formulation according to the present invention can contain active ingredients affecting the metabolism, such as antidiabetics, diuretics, antilipemics, glucocorticoids or anabolics, such as insulin, metformin, sulfonamide antidiabetics, glimepiride, pioglitazone, rosiglitazone, troglitazone, vildagliptine, sitagliptine, repaglinide, nateglinide, water- or lipid-soluble vitamins and derivatives thereof, other nutrients and essential elements, stanazolol, nandrolone, ezetimibe, a statin or a fibrate, e.g. simvastatin, lovastatin, atorvastatin, pravastatin, fluvastatin, rosuvastatin, clofibrate, fenofibrate.

The composition according to the present invention can contain an active ingredient suitable for the treatment of the diseases of the respiratory organs, such as an antihistamine, an antiallergetic, an antiasthmatic, a bronchodilator, a sympathomimetic, an antitussive or an expectorant, e.g. ephedrine, phenylephrine, oxymethazoline, xylomethazoline, naphazoline, chromoglycic acid, a selective β₂-adrenoreceptor-antagonist, a leucotriene-receptor antagonist, cetirizine, levocetirizine, chlorpyramine, loratadine, desloratadine, fexofenadine.

The active ingredient of the transdermal composition according to the present invention can be selected from pharmaceutical compounds suitable for the treatment of the muscular system, the bone-arthritic system and the locomotor system, such as an antirheumatic, spasmolytic, antiinflammatory or muscle relaxant compound and compounds effective against osteoporosis, e.g. papaverine, drotaverine, atropine, phenylbutazone, indomethacine, diclofenac, ubiprofen, ketoprofen, naproxen, flurbiprofen, celexocib, nifluminic acid, nimesulide and tolperison; alendronate, zolendronate or ibandronate. Externally useful antihistamines and wound healing agents can also be applied as an active ingredient of the present invention, e.g. dimethindene, diphenhydramine, azulene, dexpanthenol.

The composition according to the present invention can contain an active ingredient suitable for the treatment of cancers, e.g. an antitumor, biological alkylating agent (e.g. a nitrogenmustard analogs), alkylsulfonates, citotoxic antibiotics, antimetabolites, herbal alkaloids or antibodies against tumor cell proteins.

An active ingredient useful for the treatment of the sexual organs, sexual or sexually transmitted diseases can also be used in the formulation according to the present invention. Such active ingredients include sexual hormones, hormone antagonists, uterine stimulatory agents; e.g. progesteron, an ergot alkaloid, a prostaglandin, estradiol, estriol, estron and derivatives thereof, noretisterone, tibolone, clomiphene, contraceptives, e.g. progestogen, gestogen, norgestimat, etinodiol, desogestrel, levonorgestrel, medroxiprogesteron; andrological active ingredients including 4-oxoandrosten derivatives and 5-androstanon derivatives; e.g. methyltestosteron, mesterolon, cyproteron, apomorphin, alprostadil, sildenafil, alfuzosin, tamsulosin, terazosin, finasteride.

According to a further aspect of the present invention, there is provided a method for the preparation of the transdermal semisolid pharmaceutical preparation, which comprises admixing the active ingredient or mixture thereof with one or more volatile silicone and dispersing the thus obtained mixture in a cream or gel base, wherein the particles of the active ingredient coated by the volatile silicone or a mixture thereof form a separate phase in the gel, cream or ointment base whereas the coating by volatile silicone or mixture thereof is maintained after dispersing the active ingredient in the base as well.

The invention is based on the phenomenon that the solid particles of the active ingredient are coated by a layer of volatile silicone oil, which is mostly evaporated during the application. The remaining active ingredient with the remaining constituents of the formulation is absorbed rapidly due to the natural transport phenomena of the skin (diffusion, penetration, permeation). The degree of absorption is depending on the composition of the formulation. It is possible to formulate the transdermal preparation according to the present invention in a manner so that the active ingredient is able to provide its therapeutical effect on the skin. It is also possible, however, to select the constituents and especially their relative proportion in order to provide systemic effect for the subject active ingredient.

It has been found that the physical-chemical and microbiological stability of the formulation according to the present invention containing volatile silicones is improved as compared to those of formulations (in solution, emulsion or suspension form) known according to the prior art. This enhanced stability is due to the hydrophobic physical-chemical barrier effect of the silicone coating between the active ingredient and the vehicle base medium by which air and water are excluded from the active ingredient. By this separating effect, the active ingredient becomes unavailable for the mechanisms causing decomposition (e.g. hydrolysis, ionization, catalytic and autocatalytic decomposition).

The layer of silicone oils protects the active ingredient from chemical and microbiological challenge even in the case when the vehicle is aqueous and contains agents favourable for decomposition. The excess of the volatile silicone blocks the access to the active ingredient particles from microbiological agents responsible for decomposition (e.g. bacteria, fungi, molds etc.). Thus it is not necessary to use any conservant in the transdermal formulation according to the present invention.

The stability of the transdermal formulation has been tested under stability testing conditions usually applied in the pharmaceutical industry and it did not show any detectable change after five years storage.

During the application to the skin, the volatile silicones evaporate without residue and do not interact with the body. The product is essentially conservant-free from the viewpoint of the user. After application to the skin, the silicone compounds evaporate and the active ingredient and other constituents of the formulation remain on the skin surface. Subsequently these substances are absorbed from the skin. After the evaporation of the silicone matrix, the particles of the active ingredient remain on the skin surface embedded into a gel, which enhances and accelerates absorption into the deeper layers of the skin.

In the transdermal preparations according to the present invention, any type of volatile silicone can be used for the coating of particles. The most suitable siloxanes are hexamethyldisiloxane, octamethyltrisiloxane and decamethylpentacyclosiloxane. As a vehicle gel or cream base, compositions known from the art can be used. Preferably, a hydrophilic gel compositions is used.

The surprisingly advantegous properties of the formulation according to the present invention have been studied in membrane penetration tests in vitro.

The apparatus for the testing of membrane penetration comprises a penetration cell with accurately known surface area and volume having an open sample compartment, a system suitable for the control of environmental factors (air flow, temperature, air humidity, light exposure), a delivery system suitable for sustaining a flow of the acceptor Phase, a sampling and an analytical unit. The expression “open sample compartment cell” means that the sample present on the surface is not separated but in direct contact with the surrounding environment. Test disclosed in the present application have been carried out without air flow and light exposure at natural sunlight at the temperature of 32° C. The cross-sectional area of the cell at the membrane surface is exactly 10.00 cm², the volume of the cell is 3.00 cm³. During the tests, a cell at the membrane having a thickness of 30 μm was used. The test sample consisted of approx. 0.5 g portion of the transdermal formulation according to the present invention, which is transferred onto the membran located at the upper part of the cell. The membrane is intended for modelling the tested biological barrier, in this case, the skin.

The acceptor phase during the test consisted of 0.9 weight % sodium chloride solution. The acceptor phase was delivered through the penetration cell with constant flow rate of 1 ml/min. In the effluent, the concentration of a characteristic constituent of the test preparation (generally the pharmaceutically active ingredient) is determined. During the present tests, the assay is carried out by ultraviolet spectrometry using a spectrophotometer equipped with a flow cell. The measurement is continued for 6 hours. Using external calibration, the concentration profile as a function of eluted volume (proportional to elapsed time from sample application to the membrane) is determined and from these data, the amount of the characteristic constituent, e.g. the active ingredient is calculated which had penetrated the membrane during the test period. The rate of absorption is modelled by the relative amount penetrated the membrane during the test period to the total amount of the characteristic constituent of the test formulation present in the sample applied to the membrane. In those cases, when the characteristic constituent (e.g. pharmaceutically active ingredient) does not have sufficient absorption coefficient for ultraviolet detection or inteference occurs, other analytical method, e.g. methods of classical analysis or electroanalysis, e.g. iodometry, ion selective electrode etc. can be used.

During the testing of transdermal formulations according to the present invention formulated for topical use, we have found that the amount of the active ingredient penetrated the membrane did not exceeded 0.1%, thus it can be concluded that the active ingredient practically remained at the skin surface. It has been found that formulations according to the present invention exhibit topical effect when the amount of the active ingredient penetrated the membrane is in the range of 1 to 20%, preferably in the range of 7 to 20%, the most preferably, in the range of 12 to 20%. In those cases, when a transdermal formulation according to the present invention was prepared with the composition to achieve systemic effect, the amount of the active ingredient which penetrated the membran was in excess of 20%. In most cases, however, this value was between 66 to 95%. Table 1 discloses the amount of the active ingredient which had penetrated the membrane for several active ingredients and two compositions disclosed in the Examples. However, the person skilled in the art consider the properties of the active ingredient as well, which are known from the prior art.

TABLE 1

| | | | Relative |
|---|---|---|---|
| | | | Percentage of |
| | | | the active |
| | | | ingredient |
| | Concentration of the | | penetrated the |
| Active | active ingredient in | Formulation | membrane in 6 |
| ingredient | the formulation (%) | Example | hours* |
| Lidocaine base | 1.00% | 1. | 80.20% |
| Phenobarbital | 0.50% | 1. | 68.40% |
| Nifedipine | 2.00% | 1. | 94.20% |
| Econazole base | 1.00% | 2. | 0.03% |
| Acyclovir | 5.00% | 2. | 0.05% |
| Sulfadimidine | 5.00% | 1. | 72.40% |
| Sulfadimidine | 5.00% | 2. | 0.01% |
| Albumin tannate | 0.50% | 2. | 0.00% |
| Papaverine | 0.50% | 1. | 88.70% |
| Meloxicam | 1.00% | 1. | 97.60% |

*in the percentage of the amount of the active ingredient present in a sample (ca. 0.5 g)

The semisolid transdermal composition according to the present invention can be presented preferably in a form suitable to deliver dosage units of the preparation. In this case, the concentration of the active ingredient is chosen in such a manner that by one operation of the dispenser, a volume corresponding to a dosage unit of the active ingredient is delivered. Bottles equipped with a dispenser suitable for the delivery of metered dose reproducibly are known from the prior art and are commercially available. Such a method of dispensing can be well correlated to the dose present in a dosage form known from the prior art containing the corresponding amount of the active ingredient. Dosing of the formulation can also be carried out by enclosing a calibrated measuring cylinder or measuring spoon within the packaging of the formulation. Such methods for administration are known from the prior art.

The transdermal formulation according to the present invention is especially suitable for the preparation of dosage forms having high stability, good bioavailability and suitable for convenient administration containing lidocaine base, phenobarbital, econazole base, sulfadimidine, albumine tannate, papaverine, drotaverine, benzydamine, atropine base, micronized sulfur, pentosane polysulfate, troxeturine, pancreatine, neomycine, hydrocortisone, sulfamethoxazole, trimethoprim, amodazophen, novamidazophen, paracetamol, alprazolam, theophylline or caffeine as active ingredient. It can be appreciated very easily from the data of Table 1 that the same active ingredient can be formulated in a way to obtain topical effect (sulfadimidine, Formulation 2, 0.01% amount of the active ingredient penetrated the membrane) or to obtain high absorption and penetration rate, good bioavailability and thus systemic effect (Sulfadimidine, Formulation 1, 72.4% of the active ingredient penetrated the membrane) as modelled in vitro by membrane penetration experiment.

In the following examples, the composition and method of preparation of transdermal formulations according to the present invention are demonstrated without limiting the scope of protection to the disclosed compositions and methods.

The auxiliary agents referred to in the examples as “Silicon Fluid” are methylsiloxanes (hexamethyldisiloxane and/or octamethyltrisiloxane or mixtures thereof). The viscosity of the siloxane solutions mentioned in the examples are 0.65 cSt, 100 cSt or 200 cSt. These agents are commercially available.

### Example 1

### Transdermal Gel Suitable for Systemic Effect

| | | |
|---|---|---|
| Active ingredient | 0.1-2 | g |
| Silicone fluid 0.65 CST | 1.200 | g |
| Silicone fluid 100 CST | 0.400 | g |
| Carbopol 980 | 0.200 | g |
| Potassium hydroxide solution 10% | 0.290 | g |
| Hydroxypropyl-methylcellulose | 0.800 | g |
| Purified water | ad 40.00 | g |

The amount of the active ingredient is chosen according to the desired strength of the formulation or according to the dispensed volume and unit dosage.

### Example 2

### Transdermal Semisolid Formulation for Topical Use

| | | |
|---|---|---|
| Active ingredient | 0.05-1.0 | g |
| Silicone fluid 0.65 CST | 0.600 | g |
| Silicone fluid 200 CST | 0.300 | g |
| Carbopol 980 | 0.100 | g |
| Potassium hydroxide solution 10% | 0.145 | g |
| Hydroxypropyl-methylcellulose | 0.400 | g |
| Purified water | ad 20.000 | g |

The amount of the active ingredient is chosen according to the desired strength of the formulation or according to the dispensed volume and unit dosage.

### Example 3

### Method for Preparation

The compositions according to Example 1 or 2 as well as compositions having similar qualitative composition are produced by the following method.

3.1. Preparation of the suspension of the active ingredient The optionally micronized active ingredient is mixed with the silicone oils. Subsequently the mixture is homogenized using a suitable laboratory mixer, e.g. on laboratory scale, using an Ultra-Turrax mixing apparatus (4000 min^{-l}, 5 min).
3.2. Preparation of gel base

hydroxypropylcellulose is added in small proportions into water at the temperature of 25° C. and stirred until complete dissolution. Subsequently Carbopol 980 NF is added to the solution and stirred until dissolved. Thereafter the solution is neutralized using 10 weight % potassium hydroxide solution. Stirring is continued until a smooth gel state is obtained.

### 3.3. Preparation of Medicated Gel

Into the gel base prepared according to 3.2, the suspension of the active ingredient is added in small portions and homogenized.

1. Semisolid pharmaceutical preparation for transdermal use, which contains particles of at least one active ingredient coated with a volatile silicone oil or a mixture of such oils dispersed in a gel or cream base vehicle.

2. Pharmaceutical preparation according to claim 1, characterized in that the volatile silicone oil component is selected from hexamethyldisiloxane, octamethyltrisiloxane, decamethyl-pentacyclosiloxane or mixtures thereof.

3. Pharmaceutical preparation according to claim 1, characterized in that the active ingredient(s) are selected from pharmaceutically active compounds suitable for the treatment or prevention of infectious diseases, cancerous or haematological diseases, endocrinological, metabolic or nutritional diseases, diseases of the central nervous system, psychiatric, behaviourial and obsessive (viselkedesi) disorders, compulsive disorder, sexual and sexually transmitted disease, disorders or conditions related to the mental or cognitive function, neurological disorders, stroke, ophtalmological diseases, dental diseases, otolaryngological diseases, cardiovascular or cerebrovascular diseases, pulmonological diseases, gastroenterological or hepatological diseases, diseases of the bone-arthritic and the muscular system, immunological diseases, obstertic or gynaecological or andrological diseases or effective for the treatment of injuries caused by external physical effects or against external or internal parasites, insects or microbes or useful as a diagnostic or disinfectant ingredient.

4. Pharmaceutical preparation according to claim 1, wherein the vehicle is a hydrophilic gel base containing one or more gel-forming polymer, water and optionally other auxiliary agents.

5. Pharmaceutical preparation according to claim 1, characterized in that the gel-forming polymer in the vehicle is a carboxyvinyl polymer, hydroxypropylmethylcellulose or a mixture thereof.

6. Semisolid pharmaceutical preparation for transdermal use, containing 0.05-5.00 weight % pharmaceutically active ingredient coated with 0.5-10.0 weight % volatile silicone oil selected from hexamethylsiloxane, octamethyltrisiloxane, decamethylcyclo-pentasiloxane or a mixture thereof dispersed in a gel base containing 0.5-5.0 weight % hydrophylic polymer, preferably a carboxyvinyl polymer, hydroxypropyl-methylcellulose or a mixture thereof.

7. Pharmaceutical preparation according to claim 3, characterized in that the active ingredient is different from acyclovir, piroxicam, meloxicam, ibuprofen, diclofenac sodium and potassium salt, clotrimazole, bifonazole, metronidazole, nifedipine, nitroglycerol and cetirizine.

8. Pharmaceutical preparation according to claim 1 comprising lidocaine base, phenobarbital, econazole base, sulfadimidine, albumin tannate, papaverine, drotaverine, benzydamine, atropine base, micronized sulfur, pentosane polysulfate, troxerutine, pancreatine, neomycine base, hydrocortison, sulfamethoxazole, trimetoprim, amidazophene, novamidazofen, paracetamol, alprazolam, theophyilline or caffeine.

9. Pharmaceutical preparation according to claim 1 prepacked in a container suitable for metered delivery.

10. Pharmaceutical preparation according to claim 1 suitable for producing topical therapeutical effect.

11. Pharmaceutical preparation according to claim 1 suitable for producing local therapeutical effect.

12. Pharmaceutical preparation according to claim 1 suitable for producing systemic therapeutical effect.

13. Method for the preparation of the pharmaceutical preparation according to claim 1, which comprises mixing the active ingredient optionally in micronized form with a volatile silicone oil or a mixture of such oils and dispersing the thus obtained suspension in a gel or cream base in such a manner that the silicone coating forms a continous phase around the solid particles in the gel vehicle.
